(19) United States
(12) Patent Application Publication
(10) Pub. No.: US 2025/0268614 A1
(43) Pub. Date: Aug. 28, 2025

(54) SYSTEM AND METHOD FOR DETECTING AND PREVENTING ORGAN COLLAPSE DURING ENDOSCOPY

(71) Applicant: CALYXO, INC., Pleasanton, CA (US)

(72) Inventors: Matthew SERGE, Pleasanton, CA (US); Jee SHIN, San Diego, CA (US); Yash KOLAMBKAR, III, San Ramon, CA (US); David MESSINA, Rochester, NY (US)

(21) Appl. No.: 19/055,963

(22) Filed: Feb. 18, 2025

Related U.S. Application Data
(60) Provisional application No. 63/557,428, filed on Feb. 23, 2024.

(51) Int. Cl. A61B 17/22; A61B 1/00; A61B 1/015; A61B 1/307; A61B 17/00

(52) U.S. Cl. CPC A61B 17/22; A61B 1/00009; A61B 1/00119; A61B 1/015; A61B 1/307; A61B 17/00234; A61B 2017/00238; A61B 2017/00296; A61B 2017/00544; A61B 2017/00561; A61B 2017/22079; A61B 2217/005; A61B 2217/007

(57) ABSTRACT

A system and method for mitigating kidney collapse includes determining a reference value for some characteristic of a video image transmitted from an endoscope device, measuring how a new value of the same characteristic of the video image compares to the reference value, and querying the device condition to determine whether the device condition is such that there is a potential for kidney collapse. Optionally, the system and method include activating a warning related to the potential for kidney collapse and automatically reducing suction to mitigate the potential for kidney collapse.

### CROSS-REFERENCE

This application claims priority to and the benefit of provisional application Ser. No. 63/557,428, filed Feb. 23, 2024, which is incorporated herein by reference.

### FIELD

The present inventions are generally related to systems and methods for detecting the potential for organ collapse during endoscopic aspiration. More particularly, the inventions are directed to endoscopic video systems and their methods of use for preventing unwanted collapse of a kidney during aspiration of kidney stone.

### BACKGROUND

Kidney stones are a common medical problem that negatively affect millions of individuals worldwide. Kidney stones include one or more solid masses of material that are usually made of crystals and form in parts of the urinary tract including in the ureter, the kidney, and/or the bladder. Kidney stones range in size from small (less than about 1 cm) to very large (more than 4 cm) and may cause significant pain to the patient and damage to the kidney. One method of treatment for removal of kidney stones includes the use of an ureteroscope and an extraction catheter. A physician advances the ureteroscope through the ureter and into the kidney. The physician inspects the kidney with the ureteroscope, locating and counting the stones within the calyces of the kidney. A laser lithotripsy device is then inserted through the ureteroscope and is used to fragmentize the larger kidney stones into smaller pieces. The ureteroscope is then removed and an extraction catheter is introduced for removal of the fragmented and smaller, un-fragmented stones. The extraction catheter includes a vacuum lumen for removal of the stones through an aspiration port. The vacuum lumen is large in diameter to allow for the passage of the stones.

One problem associated with the above procedure is that the physician may inadvertently apply enough vacuum to collapse the kidney, which risks damaging the kidney. An extraction catheter with a large vacuum lumen can rapidly evacuate the kidney-much more quickly than a physician expects. Further, some calyces of the kidney are comparatively small and can collapse even when the physician is mindful of the aspiration rate.

The embodiments of the present inventions provide a system and method for, among other things, mitigating the risk of kidney collapse during aspiration of kidney stone from a kidney and/or the calyces of a kidney.

### SUMMARY

In accordance with an aspect of the invention, a system and method for kidney stone removal with the use of all the embodiments of the present inventions is provided.

In accordance with one embodiment, a kidney stone removal system is provided comprising an endoscopic device capable of providing suction to a kidney; an image processor; and a video display. The system determines a reference value for a characteristic of a video image transmitted from the endoscope device, measures how a new value of the characteristic of the video image compares to the reference value, and queries for a device condition to determine whether the device condition is such that there is a potential for kidney collapse. The system optionally activates a warning related to the potential for kidney collapse. The system optionally reduces suction to mitigate the potential for kidney collapse.

The endoscopic device can include irrigation lumens for irrigating the kidney and a vacuum lumen through which the suction is applied to the kidney.

The reference value can be determined at a point when the endoscopic device is positioned proximal to a kidney stone and at a distance sufficient to ensure aspiration of the kidney stone under standard suction and/or irrigation-plus-suction conditions. The new value can be determined at a time interval after the reference value is determined.

The reference value can be determined at a point when the endoscopic device is in a position within a calyx and in proximity to a kidney stone to be aspirated and prior to initiation of suction for removal of the kidney stone, and the new value is determined at a time interval after the reference value is determined or the new value is determined at a time interval after the initiation of suction for removal of the kidney stone.

The reference value can be a numerical value of the image characteristic. The image characteristic can include a degree of brightness.

The image characteristic can include a relationship between a distance of a distal end of the endoscopic device and a tissue wall of a calyx in which the endoscopic device has been inserted for removal of a kidney stone. The image characteristic includes a degree of brightness of the tissue wall of the calyx.

The system can determine the reference value and/or the new value based on a selected portion of the video image. The system can determine the reference value and/or the new value based on an entirety of the video image.

The device condition can include proximity of a distal end of the endoscopic device to a tissue wall while the device is not moving and during application of suction.

In accordance with one embodiment, a non-transitory recording medium storing a computer readable control program for detecting a potential for collapse of a kidney during a kidney stone treatment procedure with a catheter having an imaging unit is provided. The non-transitory recording medium storing a computer readable control program can cause a computer to perform determining a reference value for a characteristic of a video image transmitted from the catheter during a kidney stone removal procedure; measuring an image value of the same characteristic of the video image; comparing the reference value to the image value; querying a condition of the catheter to determine whether any difference between the reference value and the image value are occurring under a condition correlated with kidney collapse; optionally activating a warning related to the potential for kidney collapse; and optionally reducing a suction applied for kidney stone removal to mitigate the potential for kidney collapse. The reference value can be determined at a point when the catheter is positioned proximal to the kidney stone and at a distance sufficient to ensure aspiration of the kidney stone under standard suction and/or irrigation-plus-suction conditions and the image value is determined at a time interval after the reference value is determined. The reference value can be determined at a point when the catheter is in a position within a calyx and in proximity to a kidney stone to be aspirated and prior to initiation of suction for removal of the kidney stone and the image value can be determined at a time interval after the reference value is determined or after the initiation of suction for removal of the kidney stone. The reference value and image value are numerical values of the image characteristic. The image characteristic can include a degree of brightness. The image characteristic can include a relationship between a distance of a distal end of the catheter and a tissue wall of a calyx in which the catheter has been inserted for removal of the kidney stone. The image characteristic can include a degree of brightness of a tissue wall of the calyx.

In accordance with another embodiment, a method for treatment of kidney stone is provided, including positioning a catheter having an imaging unit in proximity to a kidney stone; capturing a video image where the kidney stone is located; determining a reference value for a characteristic of the video image; waiting for a period of time; determining an image value of the characteristic of the video image; comparing the reference value to the image value that is taken at a later time; querying condition of the catheter to determine whether any differences between the reference value and the image value are occurring under conditions correlated with kidney collapse; and modifying treatment based on determined risk of kidney collapse. The modifying treatment can include changing suction pressure for removal of the kidney stone.

### BRIEF DESCRIPTION OF THE DRAWINGS

The figures are not to scale.

FIG. 1 is a diagram of steps for a method for mitigating kidney collapse.

FIG. 2 is an exemplary embodiment of a system including an endoscopic or catheter device for implementing mitigation of kidney collapse.

FIG. 3 is an exemplary embodiment of a part of the system, illustrating a component of the imaging processor and other described elements.

### DETAILED DESCRIPTION OF EXEMPLARY EMBODIMENTS

The description herein relates primarily to a surgical procedure in which an endoscope, ureteroscope or catheter device (terms used interchangeably), which is connected to an image processor and a video display, is navigated into a patient's kidney for removal of one or more kidney stones. A kidney stone can refer to a stone that has been fragmented into smaller pieces by a laser or ultrasound. Kidney stone (or fragments) removal is accomplished by aspiration and can include the application of irrigation to fluidize or mobilize the kidney stone (or fragments) and the application of suction to aspirate the kidney stone (or fragments) and irrigation fluid through a vacuum channel in the endoscope (ureteroscope or catheter) to a collection chamber outside the patient.

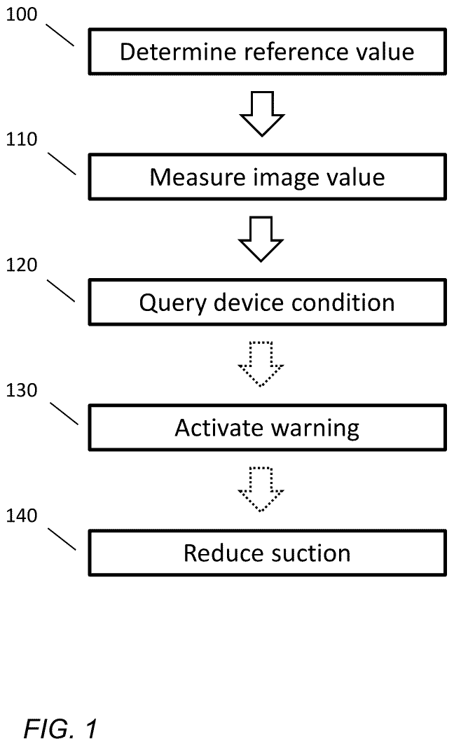

FIG. 1 is a diagram of steps in a method for mitigating kidney collapse. Step 100 involves determining a reference value for some characteristic of the video image transmitted from the endoscope. Step 110 involves measuring how a new value of the same characteristic of the video image compares to the reference value. Step 120 involves querying the device condition to determine whether the device condition is such that there is a potential for kidney collapse. Step 130 is optional. Step 130 involves activating a warning related to the potential for kidney collapse. Step 140 is optional. Step 140 involves automatically reducing suction to mitigate the potential for kidney collapse. Each of these steps is discussed in more detail below.

Referring again to step 100, in a general sense the reference value refers to a condition in which the kidney is not at risk of collapse. The reference value can be a numerical value of an image characteristic, such as brightness. Other image characteristics may also be useful as the source of the reference value. The numerical value can be determined from a selected portion of the image or the entire image.

Still discussing step 100, the numerical value of the image characteristic can be identified as the reference value at a point when the endoscope is in position to aspirate kidney stone fragments. For example, an endoscope can be navigated into a calyx of a kidney that contains kidney stone fragments to be aspirated. The endoscope is positioned proximate the kidney stone fragments—at a distance sufficient to ensure aspiration of the kidney stone fragments under standard suction and/or irrigation-plus-suction conditions. At the point just before initiating suction, the reference value of the image characteristic is determined. In particular, an image characteristic related to the distance between a distal end of the endoscope and the tissue wall of the calyx is an ideal image characteristic. The numerical value of the brightness of the tissue wall can be set as the reference value.

Now turning to step 110, an image value (or new value) is measured and compared to the reference value with the goal of determining whether that image value indicates that the kidney may be collapsing or at risk of collapse. In some circumstances, the measured image value is a value corresponding to the same image characteristic as the reference value. And the image value can be determined from a selected portion of the image or the entire image. In the example above concerning the brightness of the tissue wall as the reference value, the measured value can be the brightness of the tissue wall at a time after the reference value was determined. In one preferred embodiment, the new value can be measured at time interval(s) after suction intended for removal of the targeted stone begins. The new value can be measured 0.1 to 10 seconds after suction intended for removal of the targeted stone begins, for example about 1 second after suction begins. The time interval can scale with suction. That is, for higher suction levels, the time interval should be sooner after suction begins than with lower suction levels. This could scale with anatomy as well. For example, for higher suction power, the new value can be measured immediately or 0.1 seconds after initiating of suction. For lower suction power, the new value can be measured at around 1 to 10 seconds after initiation of suction.

Referring still to step 110, the image value can be compared to the reference value in a manner that accounts for the conditions under which the video image is captured. For example, the image capture conditions can include the lighting conditions. The endoscope will typically have integrated light sources, such as LEDs. The power levels and other conditions affecting the light output of the LEDs can augment the comparison between the image value and the reference value. For example, under relatively low light conditions, a relatively small difference between the reference value and the measured image value may be determinative of a kidney collapse. On the other hand, under relatively high light conditions, it may take a large difference between the reference value and the measured image value to determine a possibility of kidney collapse. Step 110 therefore includes in some cases an adjustment for image conditions when performing the comparison.

Still referring to step 110, in some circumstances a small or zero difference between the measured value and the reference value may be determinative of kidney collapse. For example, if the measured value stays the same as the reference value but would have been predicted to change except in circumstances where the tissue wall remains near the distal tip of the device, then that can indicate a possibility of kidney collapse. Thus, in the method disclosed herein the difference between the measured value and reference value is important, but whether it relates to kidney collapse does not always depend on whether it is large, small, or unchanged. The particular implementation of the system and method will determine how the difference between the measured value and reference value should be interpreted. For simplicity, in certain examples herein it is assumed that a large difference is indicative, but all such examples also encompass the implementations where other differences or no difference are indicative.

Turning now to step 120, the device condition is queried to determine whether the difference between the measured value and the reference value is occurring under conditions correlated with kidney collapse. There may be device conditions that generate a difference between the measured value and the reference value that indicate proximity to a tissue wall, such as during navigation of the endoscope, that do not correlate to conditions likely to be related to kidney collapse. The system can query device conditions including, but not limited to, movement of the device, location of the device distal end, rate of applied suction, rate of applied irrigation, and elapsed time of suction. Other device conditions not listed here may also be relevant conditions to query.

Still referring to step 120, one combination of device conditions that may correlate with a potential for kidney collapse includes proximity of the distal end of the device to the tissue wall while the device is not moving and while suction is being applied. Under this combination of device conditions, a significant difference between the measured value and the reference value may be an indicator for potential or imminent kidney collapse. Another combination of device conditions that may correlate with a potential for kidney collapse includes a substantial elapsed time of suction and a comparatively low rate of applied irrigation. In this combination of device conditions, the proximity of the device distal end may not be an important factor in predicting kidney collapse and a significant difference between the measured value and the reference value may be an indicator for potential or imminent kidney collapse.

Referring now to steps 130 and 140, each of steps 130 and 140 is independently chosen as optional. That is, the method can include step 130 without step 140 or step 140 without step 130. Further, steps 130 and 140 can take place sequentially or simultaneously. And steps 130 and 140 can take place in either order—step 130 before step 140 or step 140 before step 130.

Regarding step 130, a warning is activated if the combination of device conditions and the difference between the measured value and the reference value indicate that there is a potential for kidney collapse and/or that kidney collapse has begun. The warning can be internal to the system, such that it is not known to the physician, or the warning can be made known to the physician in addition to being internal to the system. The warning can be visual, audible, haptic (or tactile), or combinations thereof. Visual warnings include an indication on the video monitor, an indication on the handle on the endoscope, or both. Audible warnings include those generated on the handle, via an image processer, or on the video monitor if such monitor is equipped with audio capabilities. Haptic or tactile warnings include those generate on the handle.

Regarding step 140, suction applied to the kidney is reduced. In some cases, suction can be reduced automatically by the system via mechanisms in the device that are responsive to the output of the device condition query and/or the activated warning. In some cases, suction can be reduced at the discretion of the physician who has been warned of the potential for kidney collapse via the activated warning. In some cases, a recommendation for the degree of suction reduction can be included with the activated warning.

The system for implementing the method disclosed herein includes, but is not limited to, an endoscopic device (or ureteroscope or catheter), an image processor, and a video display. One or more of the endoscopic device, image processor, and video display can include one or more of a microprocessor, various processor modules, various computer-readable instructions, various modules of such instructions, storage elements, and transitory and/or non-transitory recording medium storing a computer readable control program for execution of the methods of the present invention, including detecting the potential for organ or kidney collapse during endoscopic aspiration.

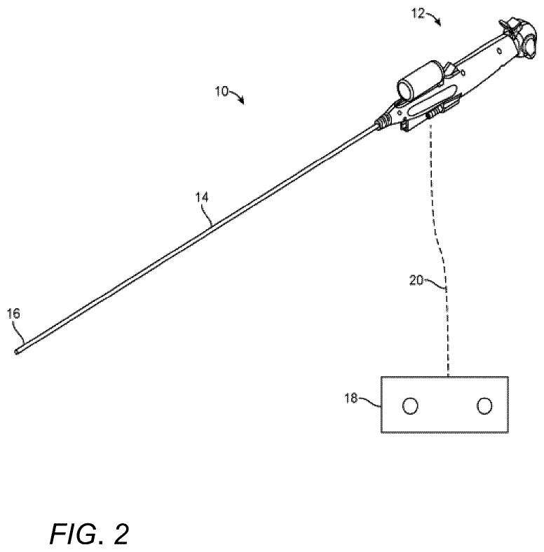

FIG. 2 illustrates an embodiment of a treatment system 10 of the present invention used for preventing unwanted collapse of a kidney during aspiration of kidney stone fragments. The system 10 can include a handle mechanism 12 from which a catheter 14 extends. The handle 12 can be configured to provide, for example, a user operable single trigger design comprising, or consisting of, two modes: an active irrigation mode only (i.e., active irrigation on/vacuum off) and an active irrigation mode in combination with a vacuum mode (i.e., active irrigation on/vacuum on). In the embodiments the handle 12 can be configured to provide, for example, a single trigger design comprising, or consisting of, three modes: passive irrigation on/active irrigation off/vacuum off; passive irrigation on/active irrigation on/vacuum off; passive irrigation on/active irrigation on/vacuum on. In an embodiment, there may be a minimum, passive amount of negative pressure even in the modes where the vacuum is off. Some aspects of the flow design allow for an uninterrupted conduit between the end of the device and the vacuum source such that there is high flow when vacuum is activated and minimal or no flow when vacuum is not activated. The catheter 14 can include various ports and lumens, including a vacuum and irrigation lumens running along the length of the catheter 14. The system 10 can also include an image processor which can include an image sensor, camera, digital visualization module, and lighting source (e.g., video chip and LED) positioned at an end 16, distal face or a distal portion of the catheter 14 for obtaining the image characteristic. A distal assembly (e.g., a nozzle) can be at the distal end 16 of the catheter 14 for irrigation and removal of the debris, with the assistance of the negative pressure applied through the vacuum lumen. The handle mechanism 12 allows the physician to hold and operate the system 10. The handle mechanism 12 can include features that allow a physical to operate various functions of the system, including the camera, vacuum pressure, the amount of irrigation and irrigation pressure, and the maneuverability of the catheter 14. For example, the handle mechanism 12 can include mechanical and electronic controls that allow the physician to adjust the amount of negative pressure, regulate the discharge of the irrigation fluid, and steer the catheter through tortuous anatomical passageways via the use of wheels and/or levers attached to cables, as is well known in the art. The system 10 can be coupled to a control unit 18 via a connector 20. The control unit 18 can control or assist in controlling aspects of the operation of the system 10. For example, the control unit 18 can control or assist in controlling visualization aspects of the system 10. The connector 20 can be a wired connection and/or a wireless connection. The control unit 18 can also include the video display, microprocessor, computer-readable instructions, and storage elements (e.g., transitory and/or non-transitory). The control unit can include a transitory and/or non-transitory recording medium storing a computer readable control program for performing the operation of the present inventions, including detecting the potential for organ or kidney collapse during endoscopic aspiration.

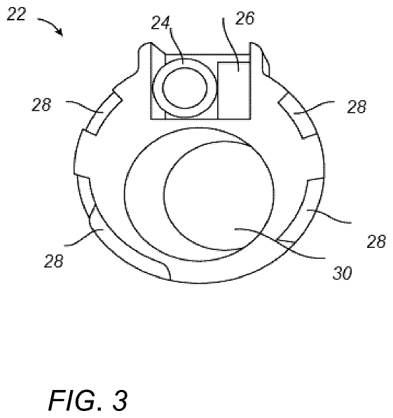

FIG. 3 illustrate an example of the distal assembly, herein shown as a nozzle tip 22. A component of the image processor such as the image sensor, camera 24, digital visualization module, and light source 26 are mounted on the nozzle tip 22. Multiple irrigation ports 28 are present as is a vacuum port 30, both of which are in communication with the irrigation lumen(s) and vacuum lumen, respectively, running along the catheter 14 body. As can be appreciated by one skilled in the art, the catheter 14 can include elongated insertable member with a working lumen or other sperate working lumens through which therapeutic or diagnostic devices can be slid. Example of such devices include, but are not limited to, lasers, sensors, and graspers.

It is understood that this disclosure, in many respects, is only illustrative of the numerous alternative device embodiments of the present inventions. Changes may be made in the details, particularly in matters of shape, size, material and arrangement of various device components without exceeding the scope of the various embodiments of the invention. Those skilled in the art will appreciate that the exemplary embodiments and descriptions thereof are merely illustrative of the invention as a whole. While several principles of the inventions are made clear in the exemplary embodiments described above, those skilled in the art will appreciate that modifications of the structure, arrangement, proportions, elements, materials and methods of use, may be utilized in the practice of the invention, and otherwise, which are particularly adapted to specific environments and operative requirements without departing from the scope of the invention. In addition, while certain features and elements have been described in connection with particular embodiments, those skilled in the art will appreciate that those features and elements can be combined with the other embodiments disclosed herein.

What is claimed is:

1. A kidney stone removal system, comprising:
an endoscopic device capable of providing suction to a kidney;
an image processor; and
a video display;
wherein the system determines a reference value for a characteristic of a video image transmitted from the endoscope device, measures how a new value of the characteristic of the video image compares to the reference value, and queries for a device condition to determine whether the device condition is such that there is a potential for kidney collapse;
wherein the system optionally activates a warning related to the potential for kidney collapse; and
wherein the system optionally reduces suction to mitigate the potential for kidney collapse.

2. The kidney stone removal system of claim 1, wherein the endoscopic device comprises irrigation lumens for irrigating the kidney and a vacuum lumen through which the suction is applied to the kidney.

3. The kidney stone removal system of claim 1, wherein the reference value is determined at a point when the endoscopic device is positioned proximal to a kidney stone and at a distance sufficient to ensure aspiration of the kidney stone under standard suction and/or irrigation-plus-suction conditions; and
the new value is determined at a time interval after the reference value is determined.

4. The kidney stone removal system of claim 1, wherein
the reference value is determined at a point when the endoscopic device is in a position within a calyx and in proximity to a kidney stone to be aspirated and prior to initiation of suction for removal of the kidney stone; and
the new value is determined at a time interval after the initiation of suction for removal of the kidney stone.

5. The kidney stone removal system of claim 1, wherein the reference value is a numerical value of the image characteristic.

6. The kidney stone removal system of claim 5, wherein the image characteristic includes a degree of brightness.

7. The kidney stone removal system of claim 5, wherein the image characteristic includes a relationship between a distance of a distal end of the endoscopic device and a tissue wall of a calyx in which the endoscopic device has been inserted for removal of a kidney stone.

8. The kidney stone removal system of claim 7, wherein the image characteristic includes a degree of brightness of the tissue wall of the calyx.

9. The kidney stone removal system of claim 1, wherein the system determines the reference value and/or the new value based on a selected portion of the video image.

10. The kidney stone removal system of claim 1, wherein the system determines the reference value and/or the new value based on an entirety of the video image.

11. The kidney stone removal system of claim 1, wherein the device condition includes proximity of a distal end of the endoscopic device to a tissue wall while the device is not moving and during application of suction.

12. A non-transitory recording medium storing a computer readable control program for detecting a potential for collapse of a kidney during a kidney stone treatment procedure with a catheter having an imaging unit, the non-transitory recording medium storing a computer readable control program causing a computer to perform:
determining a reference value for a characteristic of a video image transmitted from the catheter during a kidney stone removal procedure;
measuring an image value of the same characteristic of the video image;
comparing the reference value to the image value;
querying a condition of the catheter to determine whether any difference between the reference value and the image value are occurring under a condition correlated with kidney collapse;
optionally activating a warning related to the potential for kidney collapse; and
optionally reducing a suction applied for kidney stone removal to mitigate the potential for kidney collapse.

13. The non-transitory recording medium of claim 12, wherein
the reference value is determined at a point when the catheter is positioned proximal to the kidney stone and at a distance sufficient to ensure aspiration of the kidney stone under standard suction and/or irrigation-plus-suction conditions; and
the image value is determined at a time interval after the reference value is determined.

14. The non-transitory recording medium of claim 12, wherein
the reference value is determined at a point when the catheter is in a position within a calyx and in proximity to a kidney stone to be aspirated and prior to initiation of suction for removal of the kidney stone; and
the image value is determined at a time interval after the initiation of suction for removal of the kidney stone.

15. The non-transitory recording medium of claim 12, wherein the reference value and image value are numerical values of the image characteristic.

16. The non-transitory recording medium of claim 15, wherein the image characteristic includes a degree of brightness.

17. The kidney stone removal system of claim 15, wherein the image characteristic includes a relationship between a distance of a distal end of the catheter and a tissue wall of a calyx in which the catheter has been inserted for removal of the kidney stone.

18. The kidney stone removal system of claim 17, wherein the image characteristic includes a degree of brightness of a tissue wall of the calyx.

19. A method for treatment of kidney stone:
positioning a catheter having an imaging unit in proximity to a kidney stone;
capturing a video image where the kidney stone is located;
determining a reference value for a characteristic of the video image;
waiting for a period of time;
determining an image value of the characteristic of the video image;
comparing the reference value to the image value that is taken at a later time;
querying condition of the catheter to determine whether any differences between the reference value and the image value are occurring under conditions correlated with kidney collapse; and
modifying treatment based on determined risk of kidney collapse.

20. The method of claim 19, wherein the modifying treatment comprises changing suction pressure for removal of the kidney stone.
